(19) United States
(12) Patent Application Publication
(10) Pub. No.: US 2026/0290573 A1
(43) Pub. Date: Sep. 24, 2026

(54) METHODS AND SYSTEMS FOR SLEEP ANALYSIS

(71) Applicant: Somnology, Inc., Redwood City, CA (US)

(72) Inventors: Randol Roy Augustine SPAULDING, Menlo Park, CA (US); Melissa Susann LIM, San Francisco, CA (US); Brian Ko YAM, Portola Valley, CA (US)

(21) Appl. No.: 19/682,824

(22) Filed: May 20, 2026

Related U.S. Application Data
(63) Continuation of application No. PCT/US24/57122, filed on Nov. 22, 2024.
(60) Provisional application No. 63/602,021, filed on Nov. 22, 2023.

(51) Int. Cl. G16H 40/63; G16H 50/20

(52) U.S. Cl. CPC G16H 40/63; G16H 50/20

(57) ABSTRACT

Disclosed herein are methods and systems for suggesting an action to a user based on a sleep history of the user. The sleep history of the user may comprise a plurality of responses to a plurality of open-ended questions about the sleep quality of the user, data from a wearable device, and data from a medical device. The methods and systems disclosed herein comprise generating the action at a sleep classification system, based on the sleep history data. In some embodiments of the method, the sleep classification system comprises a machine learning model.

### CROSS-REFERENCE

This application is a continuation of International Application No. PCT/US24/57122, filed Nov. 22, 2024, which claims the benefit of U.S. Provisional Application No. 63/602,021, filed Nov. 22, 2023, each of which is entirely incorporated herein by reference for all purposes.

### BACKGROUND

An increasing number of devices are available for characterizing sleep quality. Improving a characterization of sleep quality may improve patient outcomes and provide clinical data to improve patient care. The methods and systems disclosed herein are related to a sleep classification system used to improve user sleep based on user data.

### SUMMARY

Disclosed herein are methods and systems for suggesting an action to a user based on a sleep history of the user. The sleep history of the user may comprise a plurality of responses to a plurality of open-ended questions about the sleep quality of the user, data from a wearable device, and data from a medical device. The methods and systems disclosed herein comprise generating the action at a sleep classification system, based on the sleep history data. In some embodiments of the method, the sleep classification system comprises a machine learning model.

In an aspect, disclosed herein, is a method, comprising: (a) receiving a sleep dataset, at a patient interface, wherein the sleep data set comprises at least: a plurality of user responses to a plurality of open-ended prompts corresponding to a sleep history of a user and at least one additional data modality, wherein the at least one additional data modality comprises data from a wearable sleep monitor, (b) creating a user profile at a sleep analysis model based on the sleep dataset at a plurality of instances of time, the sleep analysis model comprising a symptom classifier, (c) characterizing, at the symptom classifier, the plurality of user responses and the data from a wearable sleep monitor based at least in part on a set of sleep symptoms, to output a sleep symptom, (d) generating, at the sleep analysis model, at least one insight based on a change in the user profile over the plurality of instances of time, wherein the at least one insight is indicative of the sleep symptom and is scored based on a metric of diagnostic value, and (e) directing to a clinician interface a plurality of ranked action proposals based at least in part on the at least one insight and the metric of diagnostic value.

In some embodiments, the set of sleep symptoms, within the symptom classifier, is categorized as any one or any combination of insomnia, daytime sleepiness, daytime fatigue, abnormal breathing, snoring, waking often during sleep, movement abnormalities, restless leg syndrome, sleepwalking, sleep-eating, bedwetting, difficulty concentrating, nightmares, and needing stimulants to stay awake during daytime.

In some embodiments, a combination of symptoms of the set of sleep symptoms is indicative of a particular action.

In some embodiments, the combination of symptoms of the set of sleep symptoms is indicative of a mental health issue in relation to sleep.

In some embodiments, the mental health issue comprises PTSD.

In some embodiments, the method further comprises: classifying the plurality of user responses based at least in part on a plurality of n-grams, wherein the plurality of n-grams is connected to an insight of the at least one insight.

In some embodiments, the method further comprises: indicating at least one cause of a sleep disorder based at least in part on the plurality of n-grams.

In some embodiments, the method further comprises: soliciting at least one response from a user based at least in part on one or more of the plurality of n-grams.

In some embodiments, the plurality of open-ended prompts comprises human generated or AI-generated prompts in response to the plurality of user responses at the patient interface.

In some embodiments, the AI-generated prompts are selected from a set of follow-up prompts.

In some embodiments, the selecting from the set of follow-up prompts is based at least in part on the plurality of user responses.

In some embodiments, the method further comprises: generating the AI-generated prompts at a generative machine learning model based at least in part on the plurality of user responses.

In some embodiments, the method further comprises: receiving, at the clinician interface, the AI-generated prompts, wherein the clinician interface is configured to enable a clinician to moderate the AI-generated prompts.

In some embodiments, the method further comprises: receiving, at the patient interface, at least one moderated AI-generated prompt, wherein the at least one moderated AI-generated comprises edits or selections by the clinician at the clinician interface.

In some embodiments, the method further comprises: communicating, at the user interface the at least one moderated AI-generated prompt.

In some embodiments, the at least one insight comprises an indication that the user is healthy, or wherein the at least one insight comprises a no-insight categorization.

In some embodiments, the method further comprises: annotating, at the patient interface, at least one of the plurality of ranked action proposals, wherein the user interface is configured to generate at least one user-annotated ranked action proposal from a user annotation.

In some embodiments, the user annotation, at the patient interface, comprises edits, selections, feedback, or any combination thereof.

In some embodiments, the method further comprises: annotating, at the clinician interface, at least one of the plurality of ranked action proposals, wherein the clinician interface is configured to generate at least one clinician-annotated ranked action proposal from a clinician annotation.

In some embodiments, the clinician annotation, at the clinician interface, comprises edits, selections, feedback, or any combination thereof.

In some embodiments, the method further comprises: receiving, at the patient interface, the at least one user-annotated ranked action proposal, or the at least one clinician-annotated ranked action proposal, or any combination thereof.

In some embodiments, the method further comprises: communicating, at the patient interface, at least one of the plurality of ranked action proposals to the user, the clinician, or any combination thereof.

In some embodiments, the method further comprises: communicating, at the clinician interface, the at least one ranked action proposal to the clinician with a direction for clinician approval.

In some embodiments, the at least one ranked action proposal comprises a recommendation for a user intervention.

In some embodiments, the recommendation for the user intervention comprises at least one of a suggestion for healthy sleep behaviors or a treatment for a sleep disorder.

In some embodiments, the method further comprises: receiving a clinician approval from the direction for clinician approval and directing the at least one ranked action proposal to the user.

In some embodiments, the method further comprises: generating a text-based summary of at least one of the plurality of ranked action proposals and directing the text-based summary to the user, at the user interface.

In some embodiments, the at least one additional data modality further comprises one or more of: CPAP machine data, spirometer data, non-sleep-medical data, or sleep ranking data.

In some embodiments, the wearable sleep monitor comprises a wearable device.

In some embodiments, the wearable device is a watch or a ring.

In some embodiments, the wearable device comprises: Circul+Ring, Oura Ring, Galaxy Ring, Galaxy Watch, or Apple Watch.

In some embodiments, the data from the wearable sleep monitor comprises one or more of pulse rate, oxygen saturation, or derived sleep stage.

In some embodiments, the CPAP machine data comprises breathing rate.

In some embodiments, the sleep-ranking data comprises an indication of a quality of a user sleep during a particular sleep session.

In some embodiments, the indication of the quality of the user sleep comprises a ranking on an ordinal scale provided by the user, at the user interface.

In some embodiments, the sleep-ranking data is provided by the symptom classifier.

In some embodiments, the plurality of user responses to the plurality of open-ended prompts comprises a text-based response or a voice-based response.

In some embodiments, (d) comprises a proposal for a telehealth interaction.

In some embodiments, the method further comprises: generating a transcription of the telehealth interaction.

In some embodiments, the method further comprises: integrating the transcription of the telehealth interaction into the user profile.

In some embodiments, the method further comprises: generating a text-based summary of the telehealth interaction and integrating the text-based summary of the telehealth interaction into the user profile.

In some embodiments, the method further comprises: at (d) receiving an indication from the clinician that the at least one insight is legitimate.

In some embodiments, the method further comprises: at (d) receiving from the clinician a text-based summary of a clinician analysis.

In some embodiments, the method further comprises: integrating the text-based summary of the clinician analysis into the user profile.

In some embodiments, the sleep dataset is source agnostic.

In some embodiments, the at least one insight comprises a pattern, trend, or anomaly in a user's sleep.

In some embodiments, the method further comprises: thresholding the at least one insight based at least in part on a metric of diagnostic value.

In some embodiments, the method further comprises: updating the user profile each time a new data point is added to the sleep dataset.

In some embodiments, the plurality of ranked action proposals comprises an expiration date, wherein the expiration data indicates how long an action remains active.

In some embodiments, at (c), the symptom classifier comprises a language processing model, wherein the language processing model categorizes the plurality of user responses at least in part on the set of sleep symptoms.

In some embodiments, the language processing model comprises a BART model.

In some embodiments, at (c), the symptom classifier comprises a sleep-rating classifier, wherein the sleep-rating classifier ranks the plurality of user responses on an ordinal scale.

In some embodiments, the sleep-rating classifier further comprises a BERT model and a transcription model.

In some embodiments, the transcription model comprises a large language model.

In some embodiments, the large language model transcribes speech from the plurality of user responses in at least one language.

In another aspect, disclosed herein, is a system for sleep analysis, the system comprising: a patient interface, the patient interface configured to receive a sleep dataset, wherein the sleep data set comprises at least: a plurality of user responses to a plurality of open-ended prompts corresponding to a sleep history of a user and at least one additional data modality, wherein the at least one additional data modality comprises data from a wearable sleep monitor, a sleep analysis system, the sleep analysis system configured to create a user profile based on the sleep dataset at a first instance of time, wherein the sleep analysis system is configured to generate at least one insight based on a change in the user profile from the first instance of time to a second instance of time, and wherein the sleep analysis system comprises: a symptom classifier, wherein the symptom classifier is configured to categorize the plurality of user responses and the data from a wearable sleep monitor, and a clinician interface, the clinician interface configured to provide a plurality of ranked action proposals based at least in part on the at least one insight.

In some embodiments, the sleep history of the user comprises a set of symptoms categorized as any one or any combination of insomnia, daytime sleepiness, daytime fatigue, abnormal breathing, snoring, waking often during sleep, movement abnormalities, restless leg syndrome, sleepwalking, sleep-eating, bedwetting, difficulty concentrating, nightmares, and needing stimulants to stay awake during daytime.

In some embodiments, a combination of symptoms of the set of symptoms is indicative of a particular action.

In some embodiments, the combination of symptoms is indicative of a mental health issue in relation to sleep.

In some embodiments, the mental health issue comprises PTSD.

In some embodiments, the system further comprises: a plurality of n-grams, wherein the plurality of n-grams is connected to a particular insight, and wherein the plurality of n-grams is configured to classify the plurality of user responses.

In some embodiments, the plurality of n-grams comprises an indication of a cause of a sleep disorder.

In some embodiments, the plurality of n-grams is further configured to solicit at least one response from a user.

In some embodiments, the plurality of open-ended prompts comprises human generated or AI-generated prompts in response to the plurality of user responses at the patient interface.

In some embodiments, the AI-generated prompts are selected from a set of follow-up prompts to create a subset of AI-generated prompts.

In some embodiments, the subset of the selected follow-up prompts is based at least in part on the plurality of user responses.

In some embodiments, the system further comprises: a generative machine learning model configured to generate the AI-generated prompts based at least in part on the plurality of user responses.

In some embodiments, the clinician interface is configured to enable moderating the AI-generated prompts by a clinician to generate at least one moderated AI-generated prompt.

In some embodiments, the moderating, at the clinician interface, comprises editing or selecting the AI-generated prompts from a set of potential responses.

In some embodiments, the patient interface is configured to receive the at least one moderated AI-generated prompt.

In some embodiments, the patient interface is further configured to provide notifications of the at least one moderated AI-generated prompt to the user.

In some embodiments, the notifications comprise a push notification, a text-based notification, or a dropdown notification.

In some embodiments, the patient interface is further configured to generate at least one user-annotated ranked action proposal from a user annotation to the at least one ranked action proposal.

In some embodiments, the user annotation, at the patient interface, comprises edits, selections, feedback, or any combination thereof.

In some embodiments, the at least one insight comprises an indication that the user is healthy, or wherein the at least one insight comprises a no-insight categorization.

In some embodiments, the patient interface is further configured to enable annotating of at least one of the plurality of ranked action proposals by the user to generate at least one user-annotated ranked action proposal, and wherein the patient interface is further configured to integrate the at least one user-annotated ranked action proposal into the user profile.

In some embodiments, the at least one user-annotated ranked action proposal comprises edits, selections, feedback, or any combination thereof.

In some embodiments, the clinician interface is further configured to enable annotating of at least one of the plurality of ranked action proposals by a clinician to generate at least one clinician-annotated ranked action proposal, and wherein the clinician interface is further configured to integrate the at least one clinician-annotated ranked action proposal into the user profile.

In some embodiments, the at least one clinician-annotated ranked action proposal comprises edits, selections, feedback, or any combination thereof.

In some embodiments, the patient interface is further configured to receive the at least one of the plurality of ranked action proposals.

In some embodiments, the clinician interface is further configured to receive the at least one ranked action proposal with a direction for clinician approval.

In some embodiments, the at least one ranked action proposal comprises a recommendation for a user intervention.

In some embodiments, the recommendation for the user intervention comprises at least one of a suggestion for healthy sleep behaviors or a treatment for a sleep disorder.

In some embodiments, the patient interface is further configured to receive a clinician approval from the direction for clinician approval and to direct the at least one ranked action proposal to the user at the patient interface.

In some embodiments, the patient interface is further configured to generate a text-based summary of at least one of the plurality of ranked action proposals and to direct the text-based summary to the user at the patient interface.

In some embodiments, the at least one additional data modality further comprises one or more of: CPAP machine data, spirometer data, non-sleep-medical data, or sleep ranking data.

In some embodiments, the wearable sleep monitor comprises a wearable device.

In some embodiments, the wearable device is a watch or a ring.

In some embodiments, the wearable device comprises: Circul+Ring, Oura Ring, Galaxy Ring, Galaxy Watch, or Apple Watch.

In some embodiments, the data from the wearable sleep monitor comprises one or more of pulse rate, oxygen saturation, or derived sleep stage.

In some embodiments, the CPAP machine data comprises breathing rate.

In some embodiments, the sleep-ranking data comprises an indication of a quality of a user sleep during a particular sleep session.

In some embodiments, the indication of the quality of the user sleep comprises a ranking on an ordinal scale provided by the user.

In some embodiments, the sleep-ranking data is provided by the symptom classifier.

In some embodiments, the plurality of user responses to the plurality of open-ended prompts comprises a text-based response or a voice-based response.

In some embodiments, the plurality of ranked action proposals further comprises a suggestion for a telehealth interaction.

In some embodiments, the clinician interface is further configured to generate a transcription of the telehealth interaction.

In some embodiments, the transcription of the telehealth interaction is integrated into the user profile.

In some embodiments, the clinician interface is further configured to generate a text-based summary of the telehealth interaction and integrate the text-based summary of the telehealth interaction into the user profile.

In some embodiments, the patient interface is further configured to receive an indication from a clinician that the at least one insight is legitimate.

In some embodiments, the patient interface is further configured to receive from a clinician a text-based summary of a clinician analysis.

In some embodiments, the user profile is further configured to integrate the text-based summary of the clinician analysis.

In some embodiments, the sleep dataset is source agnostic.

In some embodiments, the at least one insight comprises a pattern, trend, or anomaly in a user's sleep.

In some embodiments, the sleep analysis system is further configured to threshold the at least one insight based at least in part on a metric of diagnostic value.

In some embodiments, the user profile further configured to update each time a new data point is added to the sleep dataset.

In some embodiments, the plurality of ranked action proposals comprises an expiration date, wherein the expiration data indicates how long an action remains active.

In some embodiments, the symptom classifier further comprises a language processing model, wherein the language processing model categorizes the plurality of user responses at least in part on the set of sleep symptoms.

In some embodiments, the language processing model comprises a BART model.

In some embodiments, the symptom classifier further comprises a sleep-rating classifier, wherein the sleep-rating classifier ranks the plurality of user responses on an ordinal scale.

In some embodiments, the sleep-rating classifier further comprises a BERT model and a transcription model.

In some embodiments, the transcription model comprises a large language model.

In some embodiments, the large language model transcribes speech from the plurality of user responses in at least one language.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 illustrates an example flowchart of the method 100, as disclosed herein.

FIG. 2 illustrates an example of the system 200, as disclosed herein.

FIG. 3 illustrates an example flowchart of the method 300, as disclosed herein.

FIG. 4 illustrates an example flowchart of the method 400, as disclosed herein.

FIG. 5 illustrates an example flowchart of the method 500, as disclosed herein.

FIG. 6 shows a computer system that is programmed or otherwise configured to implement methods provided herein.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

Certain inventive embodiments herein contemplate numerical ranges. When ranges are present, the ranges include the range endpoints. Additionally, every sub range and value within the range is present as if explicitly written out. The term “about” or “approximately” may mean within an acceptable error range for the particular value, which will depend in part on how the value is measured or determined, e.g., the limitations of the measurement system. For example, “about” may mean within 1 or more than 1 standard deviation, per the practice in the art. Alternatively, “about” may mean a range of up to 20%, up to 10%, up to 5%, or up to 1% of a given value. Where particular values are described in the application and claims, unless otherwise stated the term “about” meaning within an acceptable error range for the particular value may be assumed.

### Systems and Methods for Analyzing Sleep Symptoms

Many individuals are affected by poor sleep quality. In addition, many individuals may struggle with a diagnosed or undiagnosed sleep disorders. Diagnosing and treating sleep disorders can often be expensive, and the cost and time to seek out a clinician to receive care related to a sleep disorder may be limiting for a patient. Telehealth services and mobile device applications have emerged to connect patients struggling with poor sleep quality to clinicians. However, telehealth services and mobile device applications can lack the ability to establish a sleep history of a patient from open-ended, subjective patient logs and wearable device data, automatically and asynchronously. Further, telehealth services and mobile device applications can lack the ability to suggest sleep-related interventions to a user automatically and asynchronously, based on a change to the user's sleep history. Telehealth services and mobile device applications can also lack the ability to perform a comprehensive analysis of the user's sleep history from AI-generated insights, with expert oversight, for high-quality feedback.

Disclosed herein are systems and methods for analyzing sleep systems which address at least some of the above drawbacks.

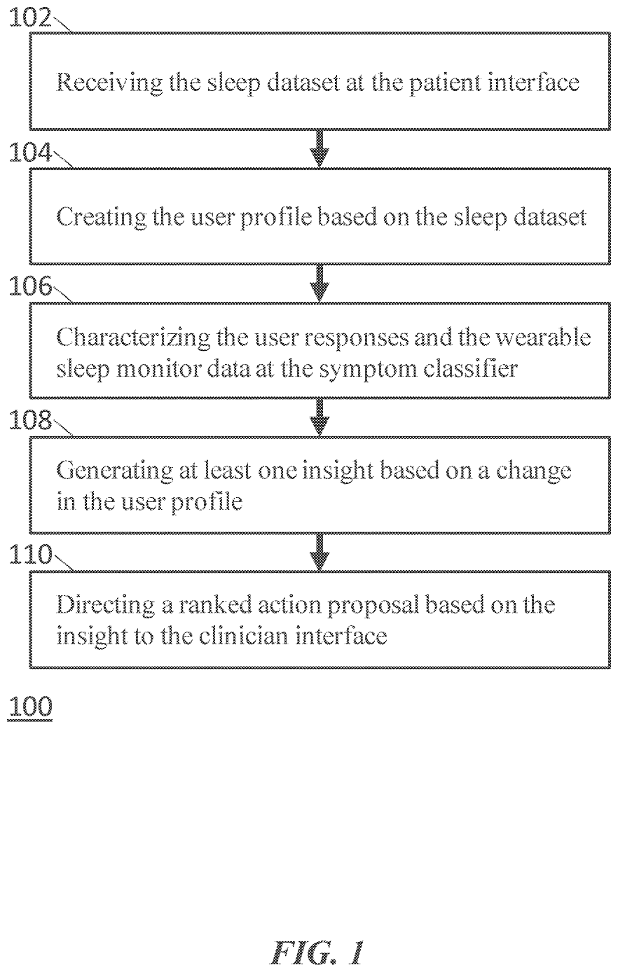

Illustrated in FIG. 1 is a flowchart of an example of a method 100. The method 100 may comprise: receiving 102 a sleep dataset, at a patient interface 202. In some cases, the sleep dataset may comprise at least: a plurality of user responses 310 to a plurality of open-ended prompts 402 corresponding to a sleep history of a user. In some cases, the plurality of open-ended prompts 402 comprises a check-in prompt. In some cases, the sleep data set may comprise at least one additional data modality, wherein the at least one additional data modality comprises data from a wearable sleep monitor 304. The method 100 may comprise creating 104 a user profile 302 at a sleep analysis model 206 based on the sleep dataset at a plurality of instances of time. In some cases, the sleep analysis model 206 comprises a symptom classifier 208. In some cases, the method 100 comprises characterizing 106, at the symptom classifier 208, the plurality of user responses 310 and the data from a wearable sleep monitor 304 based at least in part on a set of sleep symptoms, to output a sleep symptom. In some cases, the method 100 comprises generating 108, at the sleep analysis model 206, at least one insight 312 based on a change in the user profile 302 over the plurality of instances of time. In some cases, the at least one insight 312 is indicative of the sleep symptom and is scored based on a metric of diagnostic value. In some cases, the method 100 comprises directing 110, to a clinician interface 204, a plurality of ranked action proposals 314 based at least in part on the at least one insight 312 and the metric of diagnostic value.

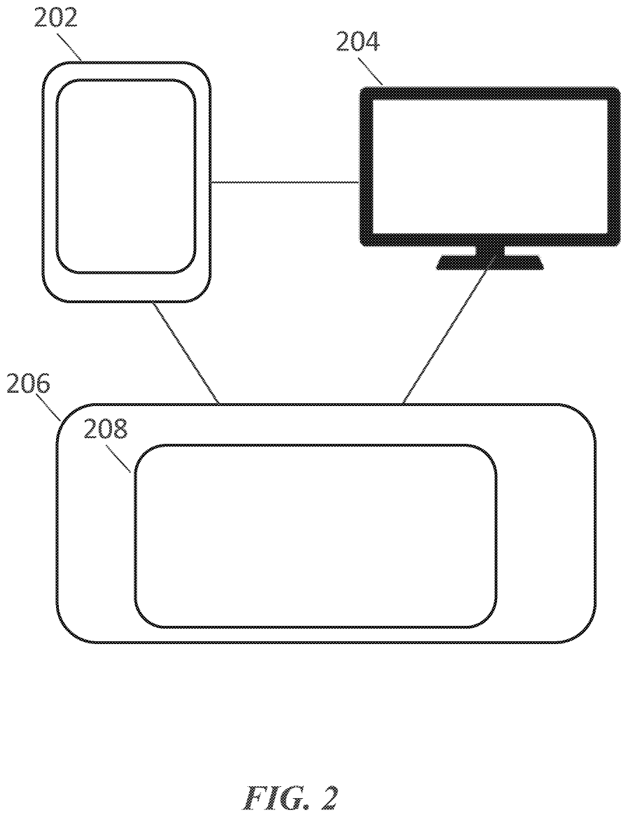

Illustrated in FIG. 2 is an example of a system for sleep analysis 200. In some cases, the system 200 may comprise a patient interface 202. In some cases, the patient interface 202 may be configured to receive a sleep dataset. In some cases, the sleep dataset comprises at least: a plurality of user responses 310 to a plurality of open-ended prompts 402 corresponding to a sleep history of a user and at least one additional data modality. In some cases, the at least one additional data modality comprises data from a wearable sleep monitor 304. In some cases, the system 200 may comprise a sleep analysis system 206. In some cases, the sleep analysis system 206 may be configured to create a user profile 302 based on the sleep dataset at a first instance of time. In some cases, the sleep analysis system is configured to generate at least one insight 312 based on a change in the user profile 302 from the first instance of time to a second instance of time. In some cases, the sleep analysis system comprises: a symptom classifier 208. In some cases, the symptom classifier is configured to categorize the plurality of user responses 310 and the data from a wearable sleep monitor 304. In some cases, the system 200 may comprise a clinician interface 204. In some cases, the clinician interface 204 may be configured to provide a plurality of ranked action proposals 314 based at least in part on the at least one insight 312.

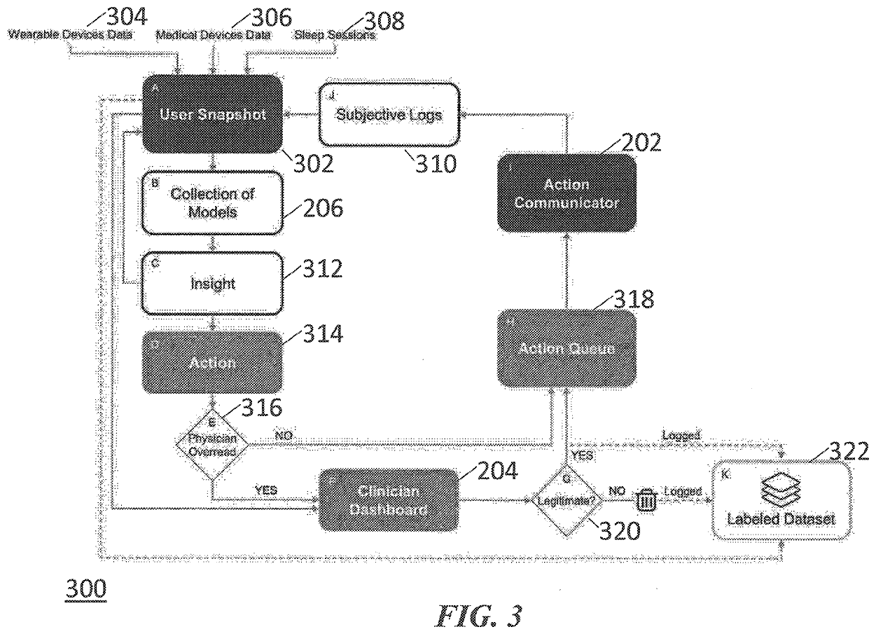

In an example, FIG. 3 provides a method 300, which illustrates an example of the method 100. The method 100 may comprise any step or sub-step of the method 300.

In some cases, the method 300 may comprise: creating a user profile 302 at a plurality of instances of time based at least in part on the sleep dataset. In some cases, the sleep dataset may comprise data from a wearable device 304, data from a medical device 306, a sleep history of the user 308, and a plurality of user responses 310 to a plurality of open-ended prompts 402 corresponding to the sleep history 308.

In some cases, the method 300 may comprise receiving the user profile 302 at the sleep analysis system 206. In some cases, the sleep analysis system comprises a symptom classifier 208. In some cases, the symptom classifier 208 may output at least one insight 312. In some cases, the at least one insight 312 may comprise a sleep symptom, based on the user profile 302, and based at least in part on a set of sleep symptoms. In some cases, the at least one insight 312 may be input into a machine learning model. In some cases, the machine learning model outputs a ranked action proposal 314 based at least in part on the at least one insight.

In some cases, the method 300 may comprise communicating the ranked action proposal 314 to a clinician, at a clinician interface 204, with a direction for clinician approval 316. In some cases, the ranked action proposal may not require clinician approval 316 and may be stored in a ranked action queue 318. In some cases, the ranked action queue 318 may prioritize actions based at least on parameters comprising complexity and severity. In some cases, ranked action proposals 314 in the ranked action queue 318 may be communicated to a user at the patient interface 202. In some cases, the ranked action proposal 314 requires a clinician approval 316. In some cases, the clinician can provide the approval 316 of the ranked action proposal 314 at the clinician interface 204. In some cases, the clinician may determine that the ranked action proposal 314 is legitimate 320, and approve the ranked action proposal, at the clinician interface 204. In some cases, approved ranked action proposals may be stored at the action queue 318, and communicated to the user at the user interface 202, based on parameters comprising complexity and severity.

In some cases, the method 300 may comprise storing the approved rank action proposal in a labeled dataset 322. In some cases, the clinician, at the clinician interface 204, may disapprove a ranked action proposal. In some cases, a disapproved ranked action proposal may be logged in the labeled dataset 322. In some cases, the sleep analysis system 206 may use the labeled dataset 322 as an input to improve insight 312 outputs and ranked action proposal 314 outputs.

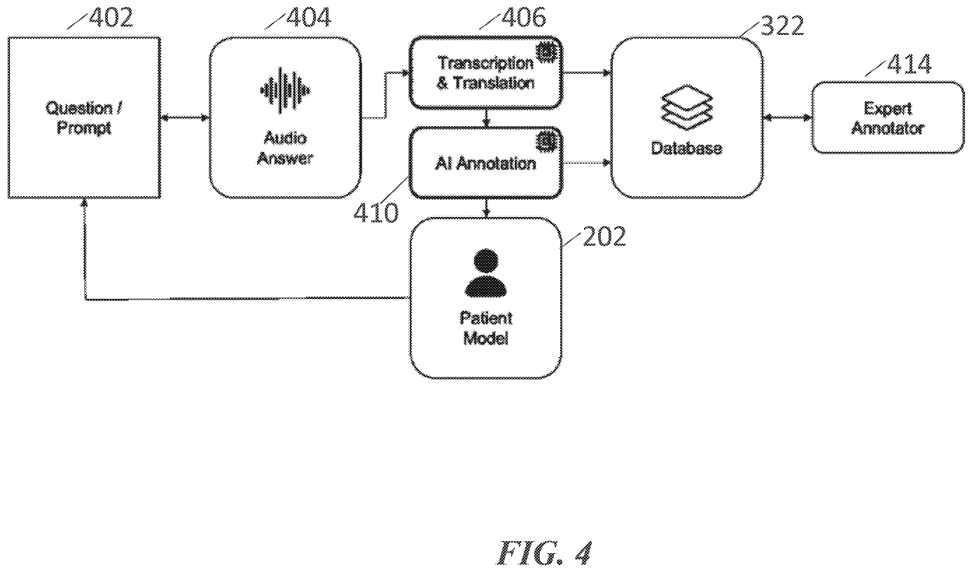

In an example, FIG. 4 provides a method 400, which illustrates an example of the method 100. The method 100 may comprise any step or sub-step of the method 400. The method 400 may comprise communicating, at the patient interface 202, a check-in prompt 402 to assess the quality of a user's sleep. In some cases, the prompt may be an open-ended prompt, wherein the open-ended prompt is generated by a generative machine learning model.

In some cases, the method 400 may comprise prompting the user to respond to the check-in prompt 402, at the patient interface 202. In some cases, responding to the check-in prompt 402 may comprise an audio-based answer 404. In some cases, the audio-based answer 404 may be translated and transcribed 406 by an automatic speech recognition system. For example, in some cases, the automatic speech recognition system may comprise Whisper. In some cases, the transcription may be stored in a dataset 322, wherein the dataset may comprise past response transcriptions from the user.

In some cases, the method 400 comprises annotating, by a machine learning model 410, the transcription 406 from the automatic speech recognition system In some cases, the annotation may comprise nuances from the speech of the user. In some cases, nuances may comprise tone, pace, rhythm, and the emotions or intentions of the user. In some cases, the transcription and the AI-annotated transcription may be integrated into the user profile 302.

In some cases, the method 400 may comprise storing the AI-annotated transcription in the dataset 322. In some cases, an expert annotator 414, such as a clinician, may review the transcriptions and the AI-annotated transcriptions, stored in the dataset. The expert annotator 414 may annotate the transcriptions 414 and the AI-annotated transcriptions 410. In some cases, annotating comprises edits, selections, feedback, or any combination thereof. In some cases, the expert-annotated transcriptions may be stored in the dataset 322.

In some cases, the method 400 may comprise inputting the dataset into the generative machine learning model. In some cases, the generative machine learning mode may output a follow-up open-ended prompt, or a new open-ended prompt, based at least on the stored transcriptions, the AI-annotated transcriptions, and the expert-annotated transcriptions. In some cases, the method 400, as described herein, may inform the generative machine learning model's decision on an appropriate open-ended prompt sequence, ensuring that a dialogue is tailored to a user profile.

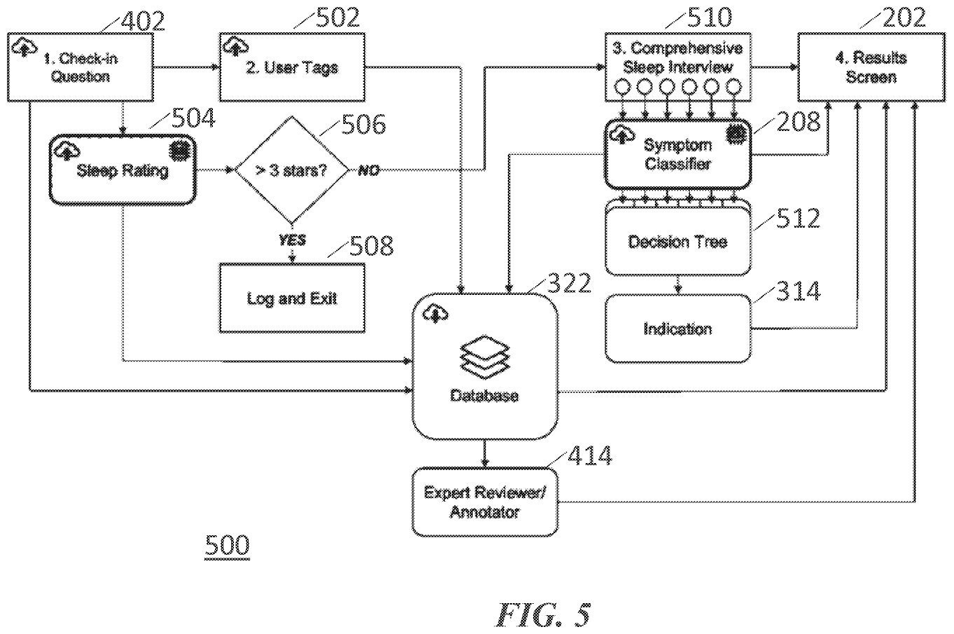

In an example, FIG. 5, provides a method 500, which illustrates an example of the method 100. The method 100 may comprise any step or sub-step of the method 500.

In some cases, the method 500 may comprise prompting the user with a check-in prompt 402. In some cases, the check-in prompt comprises a sleep rating prompt 504. In some cases, the sleep rating prompt may be communicated to the user at the user interface 202. In some cases, the sleep rating prompt 504 may comprise a prompt that solicits a response on an ordinal scale. For example, in some cases, the prompt may be “How was your sleep last night? Please rate your sleep out of 5 stars.”. For example, in some cases, if the user responds to the sleep rating prompt with a rating greater than 3 stars 506, no further prompts may be generated for the user. For example, in some cases, if the user responds to the prompt with a rating less than 3 stars 506, the user may be prompted with a comprehensive sleep interview 510.

In some cases, the comprehensive sleep interview 510 may comprise follow-up prompts. In some cases, a comprehensive sleep interview 510 may refer to a collection of exchanges completed in one continuous session with the user.

In some cases, the follow-up prompts may comprise open-ended and close-ended prompts. In some cases, the user may annotate the check-in prompt 402 or the follow-up prompts with a user annotation 502 at the patient interface 202. In some cases, the user annotation 502 may comprise feedback. In some cases, the feedback may comprise a rating of relevance of the check-in prompt 402 or the follow-up prompts, such as a thumbs-up or a thumbs down annotation. In some cases, the user annotation 502 may be stored in the labeled dataset 322.

In some cases, the method 500 may comprise inputting the plurality of user responses 310 to the comprehensive sleep interview 510 into to the symptom classifier 208. In some cases, the symptom classifier 208 may use a decision tree 512 to output a ranked action proposal 314, based at least on the at least one insight 312. In some cases, the decision tree 512 may comprise a regression tree, a categorical variable decision tree, or a random forest. Various implementations of decision trees and random forests are described herein with respect the section “Decision Tree and Random Forest.” In some cases, the method 500 may comprise communicating the ranked action proposal 314 to the user at the patient interface 202.

In some cases, the method 500 may comprise storing the outputs from the symptom classifier 208 in the labeled dataset 322. In some cases, the check-in prompt 402 may be stored in the labeled dataset 322. In some cases, the sleep rating question 504 may be stored in the labeled dataset 322. In some cases, the expert annotator 414 may annotate the information stored in the labeled dataset 322.

For example, the information stored in the labeled dataset may be communicated to the clinician at the clinician interface 204 with a direction to annotate the information stored in the labeled dataset 322. For example, in some cases, the annotated information may comprise an annotated insight or an annotated ranked action proposal. The annotated information may be communicated to the user at the patient interface 202. In some cases, the labeled dataset 322 may be input into a generative machine learning model to improve the relevance of the plurality of prompts 402. In some cases, the labeled dataset 322 may be input into the symptom classifier 208 to improve insight generation and ranked action proposal generation.

### Machine Learning

Systems and method of the present disclosure may be implemented by way of machine learning models. Machine learning models (e.g., model, ML model, AI, AI model) may have a training phase and an inference phase. During the training phase the model may learn using methods described below. During inference the machine learning model may be stopped from learning. When a model is used that has already been trained it may be called pretrained. Saying that a model is pretrained may make no assumption about the performance of the model only that it has undergone some training. Multiple rounds of training may be performed. When a pretrained model goes through a subsequent round of training it may update the model through a method such as continuous learning, fine tuning, transfer learning or other methods.

In order to train or use a model data is often input to the model. Data may be in many different classes (for example, text, image, waveform, audio, tabular, vector encoding, or noise. Some models may be configured to take multiple inputs or to give multiple outputs. Some models may take multiple inputs of multiple classes of data, for example a model which takes 3 inputs with input 1 being text, input 2 being an image, and input 3 being noise. In some cases data may comprise an example, variation or embodiment of a dataset described herein with respect to the section “Sleep Dataset.”

Data may be split into different sets. One such set may be a training set that is used during the training phase as input to the model. Optionally, other sets may be made such as a validation set which may be used during training time to provide an indication of the models performance on previously untrained data, and/or a test set which may be used after training is complete to test the trained model at inference time.

Data may be from one or more sources (such as an example, variation or embodiment of a dataset described herein with respect to the section “Sleep Dataset”). Data may be from one or more sources. Data may be multiomic. Data may be a single omics type. Data may be raw. Data may be processed. Data may be preprocessed. Data may be filtered.

Training-A machine learning model such as those disclosed here may be comprised hyperparameters (such as layer size, number of layers, choice of optimizer, learning rate. etc.), parameters (such as weights, biases, or coefficients), one or more processing steps (such as layers), and may produce one or more outputs and have one or more inputs. Hyperparameters may be optimized, in a process called hyperparameter optimization but may be set during training may not change. Parameters may be changed during training. During training the machine learning model may calculate a loss useful for calculating the error between the real output of the model and the expected output of the model (for example, labels). A loss may measure a portion of the model, such as information in the model and/or the learned distribution of samples. Some set of the model parameters may be updated based at least in part on the loss calculation. The model may perform multiple rounds, or epochs, of training wherein an input or set of inputs is given and processed by the model which may produce an output or set of outputs which may then the basis for updating the weights. The updated weights may be used in the next epoch. Some training may comprise more steps. Training may occur in different environments such as supervised, unsupervised, semi-supervised, self-supervised or some combination thereof.

In a supervised environment the expected output may be provided for each input during training. The training data may have labels associated with each sample of the training data. The labels are an indication of the desired output of the model when the corresponding input is given.

In an unsupervised method the training set does not have corresponding labels. In some cases the input is the desired output of the model and may be used in place of a label. In other cases, the desired output is communicated through a score which may be related to some other output indication.

The model may be trained using self-supervised learning (SSL). SSL may use no labels. SSL may use some labels. Self-supervised methods may generate implicit labels from the unstructured data. In SSL, tasks may fall into two categories: pretext tasks and downstream tasks. In a pretext task, SSL may be used to train an AI system to learn meaningful representations of unstructured data. Those learned representations can be subsequently used as input to a downstream task, like a supervised learning task or reinforcement learning task. The reuse of a pre-trained model on a new task is referred to as “transfer learning.”

SSL may be used in the training of a diverse array of sophisticated deep learning architectures for a variety of tasks, from transformer-based large language models (LLMs) like BERT and GPT to image synthesis models like variational autoencoders (VAEs) and generative adversarial networks (GANs) to computer vision models like SimCLR and Momentum Contrast (MoCo). These methods may use other types of learning such as semi-supervised learning, supervised learning, and/or unsupervised learning.

Semi supervised may combine unsupervised and supervised tasks by using labeled and unlabeled data. In some cases, there may be datasets where some samples are labeled and others are not. In these cases, it may be desirable to have a fully labeled dataset but producing labels for large datasets is time consuming and expensive. Semi supervised learning first trains on the labeled data of the set of data and may then be used to produce pseudo-labels, or labels that are not validated.

Labels/Ground Truth—Labels may be in various forms. Labels may be in a continuous range, for example 0 to 1. A label may use a confidence threshold. A confidence value may be associated with a label. A confidence value above a confidence threshold may be used along with the labeled data to retrain the model to improve the overall performance of the model. A label may be binary. Labels may be ordinal. Labels may be cardinal. Labels may be discrete. Labels may be vectors. Labels may be scalars. Labels may be incomplete (e.g., not all labels are present). Ground truth data may comprise data labeled from patients known to have a particular sleep disorder, particular sleep symptoms, etc.

Classification—A machine learning model may be trained as a classifier. A classifier may perform multiclass classification where more than one class is indicated. A classifier may be a multiclass multilabel, where more than one class may be output as present at one time. This may be useful in settings where classes may co-exist in the input. For example, an image segmentation model or object detection model may indicate the presence of multiple objects in an image and output an indication in its output for each of the detected objects. This may also be useful when the model is used to detect either multiple classes in the input and/or where some other label is desired such as a contextual output. For example, a sleep dataset herein may be used as an input to any example, variation, or embodiment of symptom classifier disclosed herein, for example, as described in the section “Symptom Classifier.” The symptom classifier may output a particular classification such as having a particular sleep symptom or disorder.

Regression—A machine learning model may be trained as a regression model. A regression model may be used in a predictive fashion, whereas a classifier is used to place input or portions of input into classes that are predefined. Regression models may take an input and output a continuous value as a prediction or forecast score. As an example, a regression model may take an image and predict a desired set of values describing a shape of a new object to be placed in the image. In this example the output, or a portion of the output, of a regression may be used as an input to another model.

Once training is completed, a model may be used to infer on a set of inputs. The model output may be the desired output for the use of the model or there may be some portion of the model that is used for a desired output different than the output that was used during training time. At inference time the model's weights may be static.

For example, a sleep dataset herein may be used as an input to any example, variation, or embodiment of symptom classifier disclosed herein, for example, as described in the section “Symptom Classifier.” The symptom classifier may output a predictive value, such as a score for a particular symptom or for sleep quality.

Retraining, transfer learning, fine tuning—A model may be trained. Model training may involve an optimization step wherein model parameters (such as weights or biases) may be altered based on the optimizer. Model training may involve a loss function which calculates a score based on the output of the model and the expected output of the model (such as a ground truth or labels). Model training may involve a dataset. The dataset may be split into one or more subsets. The subsets may be of different sizes. The subsets may be used for training, validation, testing or any combination thereof.

A model may be trained more than once. A model may be trained one a different dataset than was used in a previous training round (e.g., transfer learning, fine-tuning of the model, integration of the model into a larger model, continuous training, or some combination thereof). During training, whether in the first round or subsequent rounds, a subset of the model parameters may be untrainable during fine-tuning.

During fine-tuning a trained model may be trained on a different set of data, a subset of the original data or some combination thereof. Fine-tuning may cause the model to improve its performance on a given task or subtask. During transfer learning a model may be trained to improve performance on a task similar to the task the model was previously trained on. During transfer learning a model may be trained to improve performance on a task not similar to the task the model was previously trained on.

### Decision Tree and Random Forest

As described above, the machine learning model may implement a decision tree. A decision tree may be a supervised ML algorithm that can be applied to both regression and classification problems. Decision trees may mimic the decision-making process of a human brain. For example, a decision tree may grow from a root (base condition), and when it meets a condition (internal node/feature), it may split into multiple branches. The end of the branch that does not split anymore may be an outcome (leaf). A decision tree can be generated using a training data set according to the following operations: (1) Starting from a root node (the entire dataset), the algorithm may split the dataset in two branches using a decision rule or branching criterion; (2) each of these two branches may generate a new child node; (3) for each new child node, the branching process may be repeated until the dataset cannot be split any further; (4) each branching criterion may be chosen to maximize information gain (e.g., a quantification of how much a branching criterion reduces a quantification of how mixed the labels are in the children nodes). The labels may be the data or the classification that is predicted by the decision tree.

A random forest regression is an extension of the decision tree model that tends to yield more robust predictions by stretching the use of the training data partition. Whereas a decision tree may make a single pass through the data, a random forest regression may bootstrap 50% of the data (e.g., with replacement) and build many trees. Rather than using all explanatory variables as candidates for splitting, a random subset of candidate variables may be used for splitting, which may enable trees that have completely different data and different variables (hence the term random). The predictions from the trees, collectively referred to as the “forest,” may be then averaged together to produce the final prediction. Many trees (e.g., one hundred trees) may be included in a random forest model, with a number (e.g., 3, 6, 10, etc.) of terms sampled per split, a minimum of number (e.g., 1, 2, 4, 10, etc.) of splits per tree, and a minimum split size (e.g., 16, 32, 64, 128, 256, etc.). Random forests may be trained in a similar way as decision trees. Specifically, training a random forest may include the following operations: (1) select randomly k features from the total number of features; (2) create a decision tree from these k features using the same operations as for generating a decision tree; and (3) repeat the previous two operations until a target number of trees is created.

In some cases, the symptom classifier 208 may use a decision tree 512 to output a ranked action proposal 314, based at least on the at least one insight 312. In some cases, the decision tree 512 may comprise a regression tree, a categorical variable decision tree, or a random forest.

### Neural Networks

Systems and methods of the present disclosure may be implementing by way of one or more neural networks. A neural network may use artificial neurons an individual processing units. These artificial neurons may comprise at least one of an input, a set of weights, a set of biases, a summation step and an activation function (for example, rectified linear units (ReLu), exponential linear activation, sigmoid function, linear activation, leaky relu, softmax, tanh, or others) or any combination thereof. Multiple artificial neurons may be used to create a layer of neurons that takes in the same input and outputs a number of values equal to the number of neurons in that layer. A neural network may be composed of multiple layers. Layers may take as input data, or output from other layers or some other values such as a random value. Layers may be smaller, larger or the same size as the input they take. Layers may be of various types such as, but not limited to, the following layer types; dense, convolutional, pooling, recurrent, preprocessing, normalization, regularization, attention, reshaping, merging, or activation. When a layer's output is received as input by another layer the two layers are connected. Layers may be connected to any layer that follows.

Neural Network Architectures-Layer connectivity may indicate the model's architecture. Choice of a model's architecture may be directed by the task being carried out by the layer or set of layers. Layer architectures may then be described by their function. Some examples of architectures are, feed-forward networks, recurrent neural networks (RNN), long short-term memory (LSTM), echo networks, diffusion models, transformers, visual geometry group (VGG), graph neural networks (GNN), encoders, variational autoencoders (VAE), UNET, and generative adversarial networks. Networks are generally agnostic to the layer types used in them and may comprise multiple layer types. As an example, a convolutional neural network (CNN) may be a feed forward network comprising convolutional layers as well as pooling, and flattening layers, this is only an example though and CNNS may have different architectures or layer compositions. Architectures may also be combined in one model as is the case in complex models such as diffusion models and large language models (LLM).

Large language models may include models such as BART, BERT, etc. and speech recognition software (such as Whisper) A model may be a natural language processing (NLP) model. A NLP model (such as BERT and/or BART) may be trained to consider context of words. An NLP model may be bidirectional. An NLP model may be unidirectional. A NLP model may comprise an encoder. An NLP model may comprise a transformer. An NLP model may take as input a sequence of words or word tokens. An NLP model may output a sequence of words or word tokens. An NLP model may be a sequence-to-sequence model.

An NLP model may be a large language model (LLM). A large language model may predict and/or generate plausible language (for example autocomplete, BERT, ChatGPT). An LLM may estimate the probability of a token or sequence of tokens. A token may be a word and/or portion of a word. A token may be an abstraction of a word and/or portion of a word. A token may be a punctuation. A token may be an abstraction of a punctuation. A token may be a series of words. A token may be an abstraction of a series of words. A token may comprise an n-gram as described herein, where n is a number of words.

An LLM may comprise multiple types of architectures, such as but not limited to one or more attention networks, one or more transformers, one or more fully connected networks, or any combination of any network architecture. An LLM may utilize self-attention. An LLM may be fine-tuned, for example an LLM may be trained on a corpus of general text (such as text found on social media, Wikipedia, in digitized books, in digitized newspapers, on message forums, an existing library of user sleep data, etc. and fine-tuned to a specific case such as for predicting sleep symptoms and/or sleep quality herein.

### Natural Language Models

Various portions of the care system herein may be implemented by way of machine learning models comprising a natural language model. In some cases, the method 400 comprises annotating, by a machine learning model 410, the transcription 406 from the automatic speech recognition system. For example, a user may input a description of their sleep, and an NLP model may provide a summary. For example, a NLP model may provide a summary of a comprehensive sleep interview. In some cases, the NLP model may view a sleep data set and provide a summary to a user for their annotations. In some cases, the annotation may comprise nuances from the speech of the user. In some cases, nuances may comprise tone, pace, rhythm, and the emotions or intentions of the user. In some cases, the transcription and the AI-annotated transcription may be integrated into the user profile 302.

In some cases, the method 400 may comprise storing the AI-annotated transcription in the dataset 322. In some cases, an expert annotator 414, such as a clinician, may review the transcriptions and the AI-annotated transcriptions, stored in the dataset. The expert annotator 414 may annotate the transcriptions 414 and the AI-annotated transcriptions 410. In some cases, annotating comprises edits, selections, feedback, or any combination thereof. For example, an expert annotator may be a clinician. The expert annotation may comprise clinician notes on a sleep dataset. In some cases, the expert-annotated transcriptions may be stored in the dataset 322.

In some cases, the NLP model may generate a set of prompts to be used in a sleep interview. In some cases, the NLP model may be used to generate follow up questions after a starting question selected from a list.

In some cases, the NLP model may participate in a comprehensive sleep interview. The sleep interview may comprise a multi-turn question and answer dialog with the NLP model.

Further, details with respect to the interaction between the NLP model and the user are described herein with respect to the section “User Responses.”

### Sleep Dataset

Systems and methods of the present disclosure comprise a sleep dataset. In some cases, the sleep data set may be received at the patient interface 202. In some cases, the sleep dataset comprises at least a plurality of user responses 310. In some cases, the plurality of user responses may be in response to the check-in prompt 402, the comprehensive sleep interview 510, the follow-up prompts, or any combination thereof. In some cases, the sleep dataset comprises at least one additional data modality. In some cases, the at least one additional data modality comprises data from a wearable sleep monitor 304, data from a medical device 306, or any combination thereof. In some cases, the sleep dataset comprises at least the plurality of user responses 310 and the at least one additional data modality. In some cases, the sleep dataset is source agnostic. In some cases, the user profile 202 is created based on the sleep dataset. In some cases, the user profile is updated each time a new data point is added to the sleep dataset. In some cases, the user profile is updated automatically each time the new data point is added to the sleep dataset.

### User Responses

In some cases, the plurality of user responses 310 may be in response to a plurality of open-ended prompts 402. In some cases, the plurality of user responses may refer to a response provided by a user in relation to a given prompt. In some cases, the plurality of open-ended prompts may comprise the check-in prompt 402, the comprehensive sleep interview 510, or the follow-up prompts. In some cases, the plurality of user responses may be in response to open-ended prompts corresponding to a sleep history. For example, open-ended prompts may comprise subjective prompts related to the quality of the user's sleep. For example, open-ended prompts may comprise questions, such as:
How has sleep been for you lately? Feel free to describe anything that comes to mind, even if it seems insignificant.
How has your sleep been affecting your mood and day-to-day responsibilities?
Are there specific aspects of sleep that you find challenging? Perhaps falling asleep, staying asleep, or waking up earlier than you'd like?
Some people experience unique or unusual events during sleep. Has anything like that happened to you or been observed by others?
I'd like to know about how comfortable you feel during sleep. Have you experienced much discomfort or restlessness, or perhaps other sensations that seem connected to your sleep?
Life's demands may affect our sleep schedules. How regular is yours, and have you needed to adjust it often?

In some cases, open-ended prompts corresponding to a sleep history may comprise human-generated and AI-generated prompts. The AI-generated prompts may be generated at a generative machine learning model. The generative machine learning model may comprise a large language model, such as a natural language model described herein above with respect to the section “Natural Language Models.” In some cases, the comprehensive sleep interview 510 may comprise AI-generated prompts or human-generated prompts. In some cases, the follow-up prompts may comprise AI-generated prompts or human-generated prompts. In some cases, the AI-generated prompts or human-generated prompts may be selected from a set of follow-up prompts. In some cases, the set of follow-up prompts may be based at least in part on the plurality of user responses. For example, the set of follow-up prompts may comprise questions, such as:
Have you or anyone else noticed any irregularities in your breathing during sleep, like snoring or gasping?
Can you describe any recurring or particularly disturbing dreams or nightmares? How do these affect your sleep and waking life?
Do you experience sudden bouts of sleepiness during the day? How does this affect your daily activities?

In an example, the user may respond to a question, such as “Some people experience unique or unusual events during sleep. Has anything like that happened to you or been observed by others?” In some cases, a response to the question may trigger an appropriate follow-up prompt to gather additional relevant information to supplement the user response. For example, the appropriate follow-up prompt may be “Have you or anyone else noticed any irregularities in your breathing during sleep, like snoring or gasping?”

In some cases, the plurality of user responses 310 may be in response close-ended prompts comprising surveys, ordinal-point rating scales, or binary feedback. For example, ordinal-point rating scales may comprise feedback on a five-point scale. For example, binary feedback may be a thumbs-up rating or a thumbs-down rating. In some cases, the plurality of user responses may comprise responses to close-ended prompts and open-ended prompts.

In some cases, the plurality user responses to the plurality of open-ended prompts may comprise a text-based response or a voice-based response.

### Wearable Devices

In some cases, the sleep dataset comprises data from a wearable monitor 304, data from a medical device 306, or any combination thereof. In some cases, the wearable monitor comprises a wearable device.

In some cases, the wearable device is a watch or a ring. In an example, the wearable device comprises Circul+Ring, Oura Ring, Galaxy Ring, Galaxy Watch, or Apple Watch. In some cases, data from the wearable monitor comprises one or more of pulse rate, oxygen saturation, or a derived sleep stage. In some cases, the derived sleep stage is derived at least in part from the data from the wearable device. In some cases, the derived sleep stage may comprise an awake stage, a first stage of non-rapid eye movement (NREM) sleep (N1), a second stage of NREM sleep (N2), a third stage of NREM sleep (N3), a fourth stage of NREM sleep (N4), or a rapid eye movement (REM) sleep stage, or any combination thereof.

In some cases, the sleep dataset comprises data from one or more of: CPAP machine data, spirometer data, non-sleep-medical data, or sleep ranking data. In some cases, the CPAP machine data comprises breathing rate. In some cases, non-sleep-medical data comprises heart rate, steps, activity, exercise time, body temperature, or calories burned.

In some cases, the sleep-ranking data comprises an indication of a quality of a user sleep during a particular sleep session. In some cases, the indication of the quality of the user's sleep comprises the plurality of user responses 310 to a plurality of open-ended prompts 402 corresponding to the sleep history of the user. In some cases, the indication of the quality of the user's sleep comprises the plurality of user responses 310 to a plurality of closed-ended prompts corresponding to the sleep history of the user. In some cases, the indication of the quality of the user's sleep comprises a ranking on an ordinal scale provided by the user, at the patient interface. For example, the indication of the quality of the user's sleep can be prompted by a question with a 5-star scale. For example, the user may be asked “How restful was your sleep on a 5-star scale?”.

### The Sleep Analysis Model

In some cases, the sleep analysis model 206 may comprise a natural language processing (NLP) model as described herein. In some cases, the NLP model (such as BERT and/or BART) may be trained to consider context of words. In some cases, the NLP model may be bidirectional. In some cases, the NLP model may be unidirectional. In some cases, the NLP model may comprise an encoder. In some cases, the NLP model may comprise a transformer. In some cases, an NLP model may take as input a sequence of words or word tokens. For example, in some cases, the plurality of user responses 310 may be input into the NLP. In some cases, the plurality of user-responses 310 to the plurality of open-ended prompts 402 may be input to the NLP. In some cases, the NLP model may output a sequence of words or word tokens. In some cases, the NLP model may be a sequence-to-sequence model.

In some cases, the NLP model may be a large language model (LLM). In some cases, the large language model may predict and/or generate plausible language (for example autocomplete, BERT, ChatGPT). In some cases, the LLM may estimate the probability of a token or sequence of tokens. In some cases, the token may be a word and/or portion of a word. In some cases, the token may be an abstraction of a word and/or portion of a word. In some cases, the token may be a punctuation. In some cases, the token may be an abstraction of a punctuation. In some cases, the token may be a series of words. In some cases, the token may be an abstraction of a series of words.

In some cases, the LLM may comprise multiple types of architectures, such as but not limited to one or more attention networks, one or more transformers, one or more fully connected networks, or any combination of any network architecture. In some cases, the LLM may utilize self-attention. In some cases, the LLM may be fine-tuned. For example, in some cases, the LLM may be trained on the labeled dataset 322. In some cases, the sleep analysis model 206 comprises the symptom classification system 208.

### Classifying the Set of Sleep Symptoms

In some cases, the set of sleep symptoms may be categorized at the symptom classifier 208. In some cases, the symptom classifier may analyze the plurality of user responses 310 to a plurality of open-ended prompts 402 corresponding to the sleep history of the user, to categorize the set of sleep symptoms. In some cases, the symptom classifier may analyze the plurality of user responses to a plurality of close-ended prompts corresponding to the sleep history of the user, to categorize the set of sleep symptoms. In some cases, the symptom classifier may analyze the plurality of user responses to the plurality of close-ended prompts and the plurality of open-ended prompts corresponding to the sleep history of the user, to categorize the set of sleep symptoms.

In some cases, the set of sleep symptoms may be categorized at the symptom classifier with categories comprising present, absent, or unknown based at least in part of the plurality of user responses. In some cases, the symptom classifier may categorize the set of sleep symptoms comprising any one or any combination of daytime dysfunction, difficulty falling asleep, difficulty staying asleep, chronic premature awakening, sleep-disordered breathing, co-morbid cardiovascular complications, sudden and disabling daytime sleepiness, a chronic or recurrent irregular sleep schedule, an acutely irregular sleep schedule, involuntary nighttime behavior or actions, significantly disturbing nightmares, and restlessness in bed.

In some cases, the symptom classifier may categorize the set of sleep symptoms according to any one or a combination of related sleep disorders based at least in part of the set of sleep symptoms. In some cases, the related sleep disorders may comprise insomnia disorder, a sleep-related breathing disorder, hypersomnolence, a circadian rhythm sleep-wake disorder, parasomnia, or a sleep-related movement disorder. In some cases, the combination of symptoms of the set of sleep symptoms may indicate a mental health issue related to sleep. In some cases, the mental health issue related to sleep may comprise depression, an anxiety disorder, bipolar disorder, a substance use disorder, or any combination thereof. In some cases, the mental health issue related to sleep may comprise PTSD.

In some cases, the user may exhibit multiple symptoms from the set of symptoms. In some cases, the user may exhibit multiple overlapping symptoms from the set of symptoms, wherein the multiple overlapping symptoms correspond to at least one of the related sleep disorders. In some cases, a user may exhibit at least one sleep symptom from the set of sleep symptoms, wherein the at least one sleep symptom partially corresponds with at least one related sleep disorder. In some cases, the symptom classifier may categorize the at least one sleep symptom partially corresponding with the at least one related sleep disorder as a spectrum category from the at least one related sleep disorder.

For example, daytime dysfunction, difficulty falling asleep, difficulty staying asleep, and chronic premature awakening may be categorized by the symptom classifier as related to insomnia disorder. In another example, daytime dysfunction, difficulty staying asleep, and chronic premature awakening may be categorized by the symptom classifier as related to a spectrum categorization for insomnia disorder. In another example, daytime dysfunction, difficulty falling asleep, sleep-disordered breathing, co-morbid cardiovascular complications, and restlessness in bed may be categorized by the symptom classifier as related to a sleep-related breathing disorder. The sleep-related breathing disorder may comprise obstructive sleep apnea, central sleep apnea, Cheyne-Stokes respiration, or catathrenia. In another example, daytime dysfunction and sudden and disabling daytime sleepiness may be categorized by the symptom classifier as related to hypersomnolence. In another example, daytime dysfunction, a chronic or recurrent irregular sleep schedule, and an acutely irregular sleep schedule may be categorized by the symptom classifier as related to a circadian-rhythm sleep-wake disorder. In another example, involuntary nighttime behaviors or actions and significantly disturbing nightmares may be categorized by the symptom classifier as related to parasomnia. In another example, involuntary nighttime behaviors or actions and restlessness in bed may be categorized by the symptom classifier as related to a sleep-related movement disorder.

### Symptom Classifier

In some cases, the symptom classifier 208 may comprise a language processing model. In some cases, the language processing model can comprise a BART model. “BART” may stand for Bi-directional Auto-Regressive Transformer. In some cases, a BART model may use a transformer architecture. In some cases, a BART model may be bidirectional. In some cases, a BART model may comprise an encoder model and a decoder model. In some cases, a BART model may comprise a bi-directional encoder. In some cases, a BART model may use an auto-regressive decoder. In some cases, an auto-regressive model may infer a prediction based on its own previous predictions.

In some cases, the BART model can analyze the plurality of open-ended prompts 402, and analyze the plurality of user responses 310 to the plurality of open-ended prompts. In some cases, the BART model can determine if a sleep symptom is present, absent, or unknown based at least on the plurality of user responses to the plurality of open-ended prompts. In some cases, the BART model can assess the set of sleep symptoms on a scale comprising negative (−1), neutral (0), or positive (1) scores based at least in part on the plurality of responses to the plurality of open-ended questions. In some cases, a negative score can correspond with a determination of absent for a sleep symptom. In some cases, a neutral score can correspond with a determination of unknown for a sleep symptom. In some cases, a positive score can correspond with a determination of present for a sleep symptom. In some cases, the plurality of responses to the plurality of open-ended questions can be an input to the symptom classifier. In some cases, negative, neutral, or positive scores for a sleep symptom can be an output from the symptom classifier.

In some cases, the symptom classifier comprises a sleep-rating classifier, wherein the sleep-rating classifier ranks the plurality of user responses on an ordinal scale, to produce an ordinal scale ranking of the responses. In some cases, the sleep rating classifier generates sleep-ranking data. In some cases, the sleep-ranking data is provided by the symptom classifier.

In some cases, the sleep-rating classifier can comprise a BERT model and a transcription model. “BERT” may stand for Bi-directional Encoder Representations from Transformers. In some cases, a BERT model may be a language model designed for understanding the context of a word in a sentence. In some cases, a BERT model may have at least the following characteristics: it may use a transformer architecture, it may use an encoder part of a transformer, it may be bidirectional, and it may use masking for unsupervised training. In some cases, a BERT model may be a BART model that was originally trained with a more complex masking strategy. In some cases, the distinction between the BERT model and the BART model may be accuracy.

In some cases, the transcription model can comprise a large language model. In some cases, the large language model can output a transcription of a voice-based response 404 to a plurality of open-ended prompts 310. In some cases, the sleep-rating classifier can output the sleep-ranking data based on the transcription, by the transcription model, of the voice-based response, and based on the ordinal scale ranking of the responses, by the BERT model.

### Insights

In some cases, disclosed herein, is a method for generating, at the sleep analysis model 206, at least one insight 312. In some cases, the at least one insight can be based on a change in the user profile 302 over the plurality of instances of time. In some cases, the at least one insight is indicative of the sleep symptom and is scored based on a metric of diagnostic value. In some cases, the insight can comprise structured, actionable observations derived from an analysis of the user profile. In some cases, the insight can comprise patterns, trends, or anomalies in the user profile.

In some cases, the analysis of the user profile can be performed by the symptom classifier 208. In some cases, inputs to the symptom classifier can comprise the plurality of user responses 310 to the plurality of open-ended questions 402, and the at least one additional data modality. In some cases, the at least one additional data modality comprises the wearable device data 304. In some cases, outputs from the symptom classifier can comprise the at least one insight, wherein the at least one insight is indicative of the sleep symptom.

In some cases, classifying the plurality of user responses, at the symptom classifier, may be based at least in part on a plurality of n-grams. In some cases, the plurality of n-grams may be connected to an insight of the at least one insight. In some cases, the plurality of n-grams may comprise a set of co-occurring words within a snippet of text.

In some cases, the insight may be generated by a statistical model, wherein the statistical model makes a comparison of the user's most recent sleep duration to a historical sleep duration. In some cases, the comparison may comprise a percentile ranking. For example, in some cases, the user may report a sleep duration within the 10^{th} percentile of the user's historical sleep duration.

In some cases, the plurality of user responses to the plurality of open-ended questions and the plurality of close-ended questions may be input into a machine learning model, wherein the machine learning model outputs the at least one insight, and wherein the at least one insight is indicative of a sleep symptom and is scored based on a metric of diagnostic value. In some cases, the machine learning model may comprise a language model. In some cases, the language model may comprise a BERT model. For example, in some cases, a user may be prompted by an open-ended prompt to provide a description of the user's most recent sleep, as well as a sleep rating out of 5 stars for the user's most recent sleep. In some cases, the description and the sleep rating may be input to the BERT model. In some cases, the BERT model may output an insight, based at least on the description and the sleep rating. In some cases, the insight is indicative of a sleep symptom and is scored based on a metric of diagnostic value.

In some cases, the plurality of responses 310 to the plurality of open-ended prompts 402 may be input into the sleep analysis system 206. In some cases, the sleep analysis system comprises a machine learning model. In some cases, the machine learning model may map the plurality of responses into an embedding space. In some cases, the machine learning model may output at least one symptom cluster based at least on the plurality of responses.

In some cases, the machine learning model may comprise a large language model. In some cases, the plurality of responses 310 to the plurality of open-ended prompts 402 may be input into the large language model. In some cases, the large language model may generate a map of the plurality of responses in an embedding space. In some cases, the large language model may output at least one symptom cluster based at least on the map of the plurality of responses. In some cases, the map of the plurality of responses and the at least one additional data modality may be input into the symptom classifier 208, wherein the symptom classifier outputs the at least one insight.

For example, in some cases, the plurality of user responses 310 to open-ended prompts 402, such as “Have you or anyone else noticed any irregularities in your breathing during sleep, like snoring or gasping?”, and the wearable device data 304, such as oxygen desaturation index (ODI) data, may be input into the symptom classifier. For example, in some cases, symptom classifier may output a sleep symptom, wherein the sleep symptom is indicative of sleep apnea, based at least in part on the response to the prompt and the ODI data.

In some cases, the at least one insight 312 may comprise an indication that the user is healthy. In some cases, the at least one insight comprises a no-insight categorization.

### Ranked Action Proposals

In some cases, ranked action proposals 314 may comprise structured interventions or recommendations. In some cases, ranked action proposals may be generated based at least on the at least one insight 312. In some cases, ranked action proposals may be organized in a hierarchy based on parameters comprising complexity, severity, or liability of the at least one insight. In some cases, the at least one insight may be input into a machine learning model, wherein the machine learning model outputs a ranked action proposal based at least in part on the at least one insight. In some cases, the at least one insight may be thresholded based at least in part on the metric of diagnostic value.

In some cases, the combination of symptoms of the set of sleep symptoms may be indicative of a particular ranked action proposal.

In some cases, the at least one insight may be evaluated to determine if the insight warrants an action. In some cases, a plurality of insights may be associated with a similar sleep symptom. In some cases, the plurality of insights associated with the similar sleep symptom may generate a single ranked action proposal. In some cases, the ranked action proposal may comprise a suggestion for therapeutic intervention. In some cases, the suggestion for therapeutic intervention may be communicated to a clinician, at the clinician interface 204, with a direction for clinician approval. In some cases, the clinician, at the clinician interface, may approve the suggestion for therapeutic approval, wherein the approved suggestion for therapeutic approval may be communicated to the user at the patient interface 202.

In some cases, the ranked action proposal may comprise a request for additional user information. In some cases, the request for additional user information may comprise a follow-up prompt 402. In some cases, the follow-up prompt may comprise an open-ended prompt, a close-ended prompt, or any combination thereof. In some cases, the follow-up prompt may be AI-generated or human generated. In some cases, the follow-up prompt may be communicated to the user at the patient interface 202. In some cases, the user may respond to the follow-up prompt, at the patient interface. In some cases, the response to the follow-up prompt may be integrated into the user profile 302.

In some cases, the plurality of ranked action proposals may comprise an expiration date, wherein the expiration date indicates how long an action remains active. In some cases, the plurality of ranked action proposals may be stored in an action queue 318. In some cases, the plurality of ranked action proposals may be communicated in an order to the user at the patient interface 202. In some cases, the order may be determined based on parameters comprising complexity, severity, relevance, or any combination thereof.

### Clinician Interface

In some cases, the clinician interface 204 is configured to receive the AI-generated prompts 402. In some cases, the AI-generated prompts are generated by a generative machine learning model based at least on the plurality of user responses 310. In some cases, the clinician interface is configured to enable a clinician to moderate the AI-generated prompts. In some cases, moderating the AI-generated prompts by the clinician, at the clinician interface, comprises edits or selections to the AI-generated prompts. In some cases, edits may comprise amending the AI-generated prompts based at least in part on the user profile 302, to generate a moderated AI-generated prompt. In some cases, selections may comprise selecting additional AI-generated prompts or human generated prompts to generate a moderated AI-generated prompt. In some cases, the moderated AI-generated prompt may be communicated to the user, at the patient interface 202. In some cases, the moderated AI-generated prompt may be integrated into the user profile.

In some cases, the moderated AI-generated prompt may be logged in a dataset 322. In some cases, the generative machine learning model may be trained on the dataset to improve AI-generated prompt outputs.

In some cases, the at least one insight 312 may be communicated to the clinician, at the clinician interface 204, with a direction to determine if the at least one insight is legitimate 320. In some cases, the clinician interface may be configured to allow the clinician to annotate 316 the at least one insight as a legitimate insight. In some cases, the legitimate insight may be integrated into the user profile 302.

In some cases, the at least one insight may be communicated to the clinician, at the clinician interface with a direction to provide an analysis of the at least one insight. In some cases, the clinician interface may be configured to allow the clinician to provide the analysis of the at least one insight. In some cases, the analysis of the at least one insight may be integrated into the user profile. In some cases, the analysis of the at least one insight may be communicated to the user at the patient interface. In some cases, the analysis of the at least one insight may be summarized by a machine learning model, to generate a text-based summary of the analysis. In some cases, the text-based summary of the analysis may be integrated into the user profile.

In some cases, the clinician interface is configured to receive at least one of the plurality of ranked action proposals 314. In some cases, the clinician interface is configured to enable the clinician to annotate the at least one ranked action proposal. In some cases, annotating the at least one ranked action proposal by the clinician, at the clinician interface, comprises edits, selections, feedback, or any combination thereof. In some cases, edits may comprise amending the ranked action proposal based at least on the user profile, to generate an annotated ranked action proposal. In some cases, selections may comprise selecting at least one additional action proposal to include with the ranked action proposal, to generate an annotated ranked action proposal. In some cases, feedback may comprise a note or suggestion to include with the ranked action proposal, to generate an annotated ranked action proposal. In some cases, the annotated ranked action proposal may be communicated to the patient, at the patient interface, according to the methods and systems disclosed herein. In some cases, the annotated ranked action proposal may be integrated into the user profile. In some cases, the annotated ranked action proposal may be logged in a dataset 322 of annotated ranked action proposals. In some cases, the sleep analysis model may be trained on the dataset of annotated ranked action proposals to improve ranked action proposal outputs.

In some cases, the clinician interface may be configured to receive the at least one ranked action proposal with a direction for clinician approval. In some cases, the at least one ranked action proposal with the direction for clinician approval may comprise a recommendation for a user intervention. In some cases, the recommendation for the user intervention may comprise at least one of a suggestion for healthy sleep behaviors or a treatment for a sleep disorder. In some cases, the clinician interface can be configured to enable the clinician to approve the at least one ranked action proposal, to generate an approved ranked action proposal. In some cases, the approved ranked action proposal can be communicated to the user, at the patient interface. In some cases, the approved ranked action proposal can be integrated into the user profile. In some cases, the approved ranked action proposal may be logged in a dataset of approved ranked action proposals. In some cases, the sleep analysis model may be trained on the dataset of approved ranked action proposals to improve ranked action proposal outputs.

### Patient Interface

In some cases, the patient interface 202 is configured to receive at least one of the plurality of ranked action proposals 314. In some cases, the patient interface is configured to enable the user to annotate the at least one ranked action proposal. In some cases, annotating the at least one ranked action proposal by the user, at the patient interface, comprises feedback. In some cases, feedback may comprise a thumbs-up or a thumbs-down annotation. In some cases, a user-annotated ranked action proposal may be integrated into the user profile 302. In some cases, the user-annotated ranked action proposal may be logged in a dataset 322 of annotated ranked action proposals. In some cases, the sleep analysis model 206 may be trained on the dataset of user-annotated ranked action proposals to improve ranked action proposal outputs.

A patient interface may be accessed from a computer system disclosed herein, such as an example, variation or embodiment of a computer system as described herein with respect to the section “Computer System.” A patient interface may be accessed from a mobile computing device. Examples of mobile computing devices may include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants.

A patient interface may be accessed via an example, variation or embodiment of a mobile application as described herein with respect to the section “Mobile Application.” A patient interface may be accessed via an example, variation or embodiment of a web application as described herein with respect to the section “Web Application.”

### Telehealth Interactions

In some cases, the at least one insight 312 comprises a proposal for a telehealth interaction. In some cases, the proposal for the telehealth interaction may be received at the patient interface 202. In some cases, the proposal for the telehealth interaction may be received at the clinician interface 204. In some cases, the patient interface may be configured to schedule a telehealth interaction. In some cases, the clinician interface may be configured to schedule a telehealth interaction. In some cases, the patient interface may be configured to facilitate the telehealth interaction. In some cases, the clinician interface may be configured to facilitate the telehealth interaction. In some cases, the telehealth interaction may comprise an audio-based call, a video-based call, or a text-based call at the patient interface between the user and the clinician. In some cases, the telehealth interaction may comprise an audio-based interaction, a video-based interaction, or a text-based interaction at the clinician interface between the user and the clinician.

In some cases, the telehealth interaction may be transcribed by an automatic speech recognition system, at the patient interface, at the clinician interface, or both. In some cases, the automatic speech recognition system may comprise Whisper. In some cases, the patient interface may be configured to receive the transcription of the telehealth interaction. In some cases, the clinician interface may be configured to receive a transcription of the telehealth interaction. In some cases, the transcription of the telehealth interaction may be summarized by a machine learning model to generate a text-based summary of the telehealth interaction. In some cases, the machine learning model may comprise a language model. In some cases, the language model comprises a large language model. In some cases, the patient interface may be configured to receive the text-based summary of the telehealth interaction. In some cases, the clinician interface may be configured to receive the text-based summary of the telehealth interaction.

In some cases, the transcription of the telehealth interaction may be integrated into the user profile. In some cases, the text-based summary of the telehealth interaction may be integrated into the user profile.

A clinician interface may be accessed from a computer system disclosed herein, such as an example, variation or embodiment of a computer system as described herein with respect to the section “Computer System.” A clinician interface may be accessed from a mobile computing device. Examples of mobile computing devices may include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants.

A clinician interface may be accessed via an example, variation or embodiment of a mobile application as described herein with respect to the section “Mobile Application.” A clinician interface may be accessed via an example, variation or embodiment of a web application as described herein with respect to the section “Web Application.”

### Computer Systems

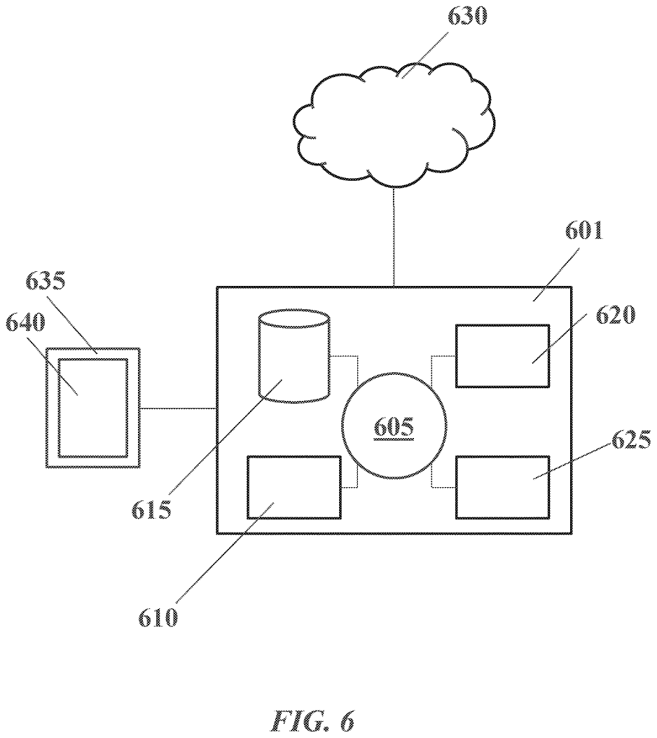

The present disclosure provides computer systems that are programmed to implement methods 100, 300, 400, and 500 of the disclosure. FIG. 6 shows a computer system 601 that is programmed or otherwise configured to enable communication between the patient interface 202, the clinician interface 204, and the sleep analysis system 206. In some cases, the communication can comprise sending and receiving data. In some cases, communication can comprise a telehealth interaction. The computer system 601 may regulate various aspects of the methods and systems of the present disclosure, such as, for example, enabling the symptom classifier 208. For example, the computer system 601 may input the plurality of user responses 310, and the wearable device data 304 into the symptom classifier. In some cases, the computer system 601 may enable the generative machine learning model to generate AI-generated prompts, based at least in part on the user snapshot 302. In some cases, the computer system 601 can enable updating the user profile each time a new data point is added to the sleep dataset. In some cases, the computer system 601 can enable the sleep analysis model to generate the at least one insight 312, based at least in part on the user profile. In some cases, the computer system can enable the sleep analysis model to output ranked action proposals 314, based at least in part on the at least one insight. The computer system 601 may be an electronic device of a user or a computer system that is remotely located with respect to the electronic device. The computer system 601 may be an electronic device of the clinician or a computer system that is remotely located with respect to the electronic device. The electronic device may be a mobile electronic device.

The computer system 601 includes a central processing unit (CPU, also “processor” and “computer processor” herein) 605, which may be a single core or multi core processor, or a plurality of processors for parallel processing. The computer system 601 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 may be a data storage unit (or data repository) for storing data. The computer system 601 may be operatively coupled to a computer network (“network”) 630 with the aid of the communication interface 620. The network 630 may be the Internet, an internet and/or extranet, or an intranet and/or extranet that is in communication with the Internet. The network 630 in some cases is a telecommunication and/or data network. The network 630 may include one or more computer servers, which may enable distributed computing, such as cloud computing. The network 630, in some cases with the aid of the computer system 601, may implement a peer-to-peer network, which may enable devices coupled to the computer system 601 to behave as a client or a server. In some cases, the peer-to-peer network may comprise the electronic device of the user, and the electronic device of the clinician.

The CPU 605 may execute a sequence of machine-readable instructions, which may be embodied in a program or software. The instructions may be stored in a memory location, such as the memory 610. The instructions may be directed to the CPU 605, which may subsequently program or otherwise configure the CPU 605 to implement methods of the present disclosure. Examples of operations performed by the CPU 605 may include fetch, decode, execute, and writeback.

The CPU 605 may be part of a circuit, such as an integrated circuit. One or more other components of the system 601 may be included in the circuit. In some cases, the circuit is an application specific integrated circuit (ASIC).

The storage unit 615 may store files, such as drivers, libraries, and saved programs. The storage unit 615 may store user data, e.g., user preferences and user programs. The computer system 601 in some cases may include one or more additional data storage units that are external to the computer system 601, such as located on a remote server that is in communication with the computer system 601 through an intranet or the Internet.

The computer system 601 may communicate with one or more remote computer systems through the network 630. For instance, the computer system 601 may communicate with a remote computer system of a user (e.g., a personal computer or a mobile device). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user may access the computer system 601 via the network 630.

Methods as described herein may be implemented by way of machine (e.g., computer processor) executable code stored on an electronic storage location of the computer system 601, such as, for example, on the memory 610 or electronic storage unit 615. The machine executable or machine readable code may be provided in the form of software. During use, the code may be executed by the processor 605. In some cases, the code may be retrieved from the storage unit 615 and stored on the memory 610 for ready access by the processor 605. In some situations, the electronic storage unit 615 may be precluded, and machine-executable instructions are stored on memory 610.

The code may be pre-compiled and configured for use with a machine having a processer adapted to execute the code, or may be compiled during runtime. The code may be supplied in a programming language that may be selected to enable the code to execute in a pre-compiled or as-compiled fashion.

Aspects of the systems and methods provided herein, such as the computer system 601, may be embodied in programming. Various aspects of the technology may be thought of as “products” or “articles of manufacture” typically in the form of machine (or processor) executable code and/or associated data that is carried on or embodied in a type of machine readable medium. Machine-executable code may be stored on an electronic storage unit, such as memory (e.g., read-only memory, random-access memory, flash memory) or a hard disk. “Storage” type media may include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the datasets, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 may include or be in communication with an electronic display 635 that comprises a user interface (UI) 640 for providing, for example, the plurality of prompts 402 at the patient interface 202, the plurality of insights 312 at the patient interface, or the plurality of ranked action proposals 314 at the patient interface. The computer system 601 may include or be in communication with an electronic display 635 that comprises a user interface (UI) 640 for providing, for example, the plurality of prompts 402 at the clinician interface 204, the plurality of insights 312 at the clinician interface, or the plurality of ranked action proposals 314 at the clinician interface. Examples of UI's include, without limitation, a graphical user interface (GUI) and web-based user interface.

Methods and systems of the present disclosure may be implemented by way of one or more algorithms. An algorithm may be implemented by way of software upon execution by the central processing unit 605. The algorithm may, for example, comprise the generative machine learning model. The algorithm may, for example, comprise the sleep system model 206. The algorithm may, for example, comprise the sleep rating classifier 208. The algorithm may, for example, comprise the speech recognition system. The algorithm may, for example, comprise the transcription model 406. The algorithm may, for example, comprise the language model.

### Computer Program

In some embodiments, the platforms, systems, media, and methods disclosed herein include at least one computer program, or use of the same. A computer program includes a sequence of instructions, executable in the connected device's CPU, written to perform a specified task. Computer readable instructions may be implemented as program modules, such as functions, objects, Application Programming Interfaces (APIs), data structures, and the like, that perform particular tasks or implement particular abstract data types. In light of the disclosure provided herein, those of skill in the art will recognize that a computer program may be written in various versions of various languages.

The functionality of the computer readable instructions may be combined or distributed as desired in various environments. In some embodiments, a computer program comprises one sequence of instructions. In some embodiments, a computer program comprises a plurality of sequences of instructions. In some embodiments, a computer program is provided from one location. In other embodiments, a computer program is provided from a plurality of locations. In various embodiments, a computer program includes one or more software modules. In various embodiments, a computer program includes, in part or in whole, one or more web applications, one or more mobile applications, one or more standalone applications, one or more web browser plug-ins, extensions, add-ins, or add-ons, or combinations thereof.

### Mobile Application

In some embodiments, a computer program includes a mobile application provided to a mobile connected device. In some embodiments, the mobile application is provided to a mobile connected device at the time it is manufactured. In other embodiments, the mobile application is provided to a mobile connected device via the computer network described herein.

In view of the disclosure provided herein, a mobile application is created by techniques known to those of skill in the art using hardware, languages, and development environments known to the art. Those of skill in the art will recognize that mobile applications are written in several languages. Suitable programming languages include, by way of non-limiting examples, C, C++, C#, Objective-C, Java™, Javascript, Pascal, Object Pascal, Python™, Ruby, VB.NET, WML, and XHTML/HTML with or without CSS, or combinations thereof.

Suitable mobile application development environments are available from several sources. Commercially available development environments include, by way of non-limiting examples, AirplaySDK, alcheMo, Appcelerator®, Celsius, Bedrock, Flash Lite, .NET Compact Framework, Rhomobile, and WorkLight Mobile Platform. Other development environments are available without cost including, by way of non-limiting examples, Lazarus, MobiFlex, MoSync, and Phonegap. Also, mobile device manufacturers distribute software developer kits including, by way of non-limiting examples, iPhone and iPad (iOS) SDK, Android™ SDK, BlackBerry® SDK, BREW SDK, Palm® OS SDK, Symbian SDK, webOS SDK, and Windows® Mobile SDK.

Those of skill in the art will recognize that several commercial forums are available for distribution of mobile applications including, by way of non-limiting examples, Apple® App Store, Google® Play, Chrome WebStore, BlackBerry® App World, App Store for Palm devices, App Catalog for webOS, Windows® Marketplace for Mobile, Ovi Store for Nokia® devices, Samsung® Apps, and Nintendo® DSi Shop.

### Web Application

In some embodiments, a computer program includes a web application. In light of the disclosure provided herein, those of skill in the art will recognize that a web application, in various embodiments, utilizes one or more software frameworks and one or more database systems. In some embodiments, a web application is created upon a software framework such as Microsoft® .NET or Ruby on Rails (RoR). In some embodiments, a web application utilizes one or more database systems including, by way of non-limiting examples, relational, non-relational, object oriented, associative, and XML database systems. In further embodiments, suitable relational database systems include, by way of non-limiting examples, Microsoft® SQL Server, mySQL™, and Oracle®. Those of skill in the art will also recognize that a web application, in various embodiments, is written in one or more versions of one or more languages. A web application may be written in one or more markup languages, presentation definition languages, client-side scripting languages, server-side coding languages, database query languages, or combinations thereof. In some embodiments, a web application is written to some extent in a markup language such as Hypertext Markup Language (HTML), Extensible Hypertext Markup Language (XHTML), or eXtensible Markup Language (XML). In some embodiments, a web application is written to some extent in a presentation definition language such as Cascading Style Sheets (CSS). In some embodiments, a web application is written to some extent in a client-side scripting language such as Asynchronous Javascript and XML (AJAX), Flash® Actionscript, Javascript, or Silverlight®. In some embodiments, a web application is written to some extent in a server-side coding language such as Active Server Pages (ASP), ColdFusion®, Perl, Java™, JavaServer Pages (JSP), Hypertext Preprocessor (PHP), Python™, Ruby, Tcl, Smalltalk, WebDNA®, or Groovy. In some embodiments, a web application is written to some extent in a database query language such as Structured Query Language (SQL). In some embodiments, a web application integrates enterprise server products such as IBM® Lotus Domino®. In some embodiments, a web application includes a media player element. In various further embodiments, a media player element utilizes one or more of many suitable multimedia technologies including, by way of non-limiting examples, Adobe® Flash®, HTML 5, Apple® QuickTime®, Microsoft® Silverlight®, Java™, and Unity®.

### Web Browser Plug-in

In some embodiments, the computer program includes a web browser plug-in (e.g., extension, etc.). In computing, a plug-in is one or more software components that add specific functionality to a larger software application. Makers of software applications support plug-ins to enable third-party developers to create abilities which extend an application, to support easily adding new features, and to reduce the size of an application. When supported, plug-ins enable customizing the functionality of a software application. For example, plug-ins are commonly used in web browsers to play video, generate interactivity, scan for viruses, and display particular file types. Those of skill in the art will be familiar with several web browser plug-ins including, Adobe® Flash® Player, Microsoft® Silverlight®, and Apple® QuickTime®. In some embodiments, the toolbar comprises one or more web browser extensions, add-ins, or add-ons. In some embodiments, the toolbar comprises one or more explorer bars, tool bands, or desk bands.

In view of the disclosure provided herein, those of skill in the art will recognize that several plug-in frameworks are available that enable development of plug-ins in various programming languages, including, by way of non-limiting examples, C++, Delphi, Java™ PHP, Python™, and VB .NET, or combinations thereof.

Web browsers (also called Internet browsers) are software applications, designed for use with network-connected devices, for retrieving, presenting, and traversing information resources on the World Wide Web. Suitable web browsers include, by way of non-limiting examples, Microsoft® Internet Explorer®, Mozilla® Firefox®, Google® Chrome, Apple® Safari®, Opera Software® Opera®, and KDE Konqueror. In some embodiments, the web browser is a mobile web browser. Mobile web browsers (also called mircrobrowsers, mini-browsers, and wireless browsers) are designed for use on mobile connected devices including, by way of non-limiting examples, handheld computers, tablet computers, netbook computers, subnotebook computers, smartphones, music players, personal digital assistants (PDAs), and handheld video game systems. Suitable mobile web browsers include, by way of non-limiting examples, Google® Android® browser, RIM BlackBerry® Browser, Apple® Safari®, Palm® Blazer, Palm® WebOS® Browser, Mozilla® Firefox® for mobile, Microsoft® Internet Explorer® Mobile, Amazon® Kindle® Basic Web, Nokia® Browser, Opera Software® Opera® Mobile, and Sony® PSP™ browser.

### Standalone Application

In some embodiments, a computer program includes a standalone application, which is a program that is run as an independent computer process, not an add-on to an existing process, e.g., not a plug-in. Those of skill in the art will recognize that standalone applications are often compiled. A compiler is a computer program(s) that transforms source code written in a programming language into binary object code such as assembly language or machine code. Suitable compiled programming languages include, by way of non-limiting examples, C, C++, Objective-C, COBOL, Delphi, Eiffel, Java™, Lisp, Python™, Visual Basic, and VB .NET, or combinations thereof. Compilation is often performed, at least in part, to create an executable program. In some embodiments, a computer program includes one or more executable complied applications.

### Software Modules

In some embodiments, the platforms, systems, media, and methods disclosed herein include software, server, and/or database modules, or use of the same. In view of the disclosure provided herein, software modules are created by techniques known to those of skill in the art using machines, software, and languages known to the art. The software modules disclosed herein are implemented in a multitude of ways. In various embodiments, a software module comprises a file, a section of code, a programming object, a programming structure, or combinations thereof. In further various embodiments, a software module comprises a plurality of files, a plurality of sections of code, a plurality of programming objects, a plurality of programming structures, or combinations thereof. In various embodiments, the one or more software modules comprise, by way of non-limiting examples, a web application, a mobile application, and a standalone application. In some embodiments, software modules are in one computer program or application. In other embodiments, software modules are in more than one computer program or application. In some embodiments, software modules are hosted on one machine. In other embodiments, software modules are hosted on more than one machine. In further embodiments, software modules are hosted on cloud computing platforms. In some embodiments, software modules are hosted on one or more machines in one location. In other embodiments, software modules are hosted on one or more machines in more than one location.

### EXAMPLES

### Example 1

### Training the Symptom Classifier

The symptom classifier underwent pretraining for summarizing conversations via its encoder-decoder architecture; however, the decoder was removed, and only the encoder was utilized. The term “pretrained” may describe a model that has been trained by an external party and made publicly available. A pretrained model may be provided through various distribution platforms, such as for example HuggingFace.

Utilizing the pretrained portions of the BART model ensured it could understand the context of sleep symptoms as described across the input sequence. This comprehension allowed the connected classification head, which was linked to the output encodings, to precisely forecast symptom categories. It can consider the various ways in which a user might convey pertinent information. To enhance its proficiency in symptom classification, the BART model underwent further training and fine-tuning on a dataset containing labeled symptom descriptions, thereby learning to correlate descriptions with symptom categories with high accuracy. “Training” describes the process of determining improved parameters comprising a model. Training may describe determining improved weights and biases. Training may describe an iterative process of making incremental adjustments to the model's parameters. Training may comprise using batches of data, aiming to enhance performance based on a specific objective function.

The model was initialized using weights from a public HuggingFace repository, which had fine-tuned the BART model for summarizing conversations. HuggingFace. co serves as one example of a distribution platform for various models, particularly language models. A language model may refer to a model that estimates the probability of a token or sequence of token or sequence of tokens occurring in a longer sequence of tokens. In the context of a language model, a token may refer to a unit of a textual input. A token may be a word, a character (e.g., a letter), subwords (e.g., a prefix, a suffix, etc.), or a sequency of words.

Each training step involved a prompt-answer exchange corresponding to a specific 12×3 trinary classification vector. To determine the optimal training environment and configuration, a hyperparameter search was conducted using Tree-structured Parzen Estimation (TPE), taking into account the constraints imposed by the hardware, which included two Nvidia V100 GPUs. A hyperparameter may be a variable that is tuned or adjusted during successive runs of training a model. In contrast parameters may be the various weights and biases that the model learns during training. Hyperparameters are parameters of a model that are not learned during training. Hyperparameters may comprise a number of epochs, a learning rate, a batch size, etc. Hyperparameters may be tuned by a tuning services, manually, etc. A hyperparameter search is one example of a method of hyperparameter tuning. Hyperparameter search may involve a comprehensive exploration of all hyperparameter combinations (grid search), a selective approach (Bayesian search), or another search to identify more effective combinations of hyperparameter values.

The ideal training duration was identified as four epochs, with a batch size of 32 and a learning rate of 5e-5. The term “epoch” may refer to a cycle of training. For example, a single epoch is completed when the model has trained on every data point in the dataset once. Early stopping was also implemented; this meant that if no improvement was observed during validation at the end of each epoch, and if this lack of improvement persisted for two consecutive epochs, the training would be terminated.

To develop a machine learning model proficient in detecting sleep-related symptoms from conversations, a comprehensive approach to data curation and augmentation, emphasizing both quality and diversity was utilized. The training set comprised of thousands of input-output pairs. A “training set” may be a subset of a dataset reserved for training a model. A training set may be a relatively large portion of the dataset, often comprising 60-80% of the total dataset used to train the model.

In these pairs, conversation snippets served as inputs, while the identified symptoms are the outputs. The size and diversity of this dataset were critical, ensuring that the model could adeptly navigate through various exceptions and idioms in real conversations.

To further enrich the training set and introduce additional diversity, the training set incorporated a synthetic dataset, which constituted 20% of the training data. This dataset was generated by instructing GPT-4 to act as a user with a predefined set of symptoms, offering a wider range of scenarios and expressions for the model to learn from. The responses from GPT-4 were used to bootstrap the dataset and establish a broader baseline for discussing underrepresented sleep symptoms.

The training set also comprised a patient dataset, comprising real interactions with human patients discussing their sleep experiences. The patient data set exposed the sleep classifier to a wide range of symptoms and expressions, vital for accurate detection and understanding. During the training phase, 10% of the patient dataset was set aside as the validation set. A validation set may be a subset of a dataset used for initial evaluation against a trained model. A validation set may be used to determine when the model is adequately trained and ready for testing. A trained model may be evaluated against the validation set several times before evaluation the model against a test set. A validation set may make up about 10% of the total data. A test set may be a subset of a dataset reserved for testing a trained model. The test set may be a subset of data used to evaluate the performance of the model. A test set may constitute 10¬20% of the total data. A dataset may be divided into a training set, a test set, and a validation set. In some cases, a single example does not belong to two or more of the training set, the test set, and the validation set.

The final step in evaluating the model's capabilities was the test set, which was exclusively composed of patient data. This set served as an unbiased benchmark to assess the model's proficiency in symptom detection, offering a clear measure of its real-world applicability. Throughout this process, overfitting a model was avoided—where a model might, for instance, memorize inputs too strictly, associating terms like “groggy” with daytime dysfunction without truly understanding the underlying relationships. Overfitting may describe a phenomena that occurs when a model memorizes input data rather than learning general relationships between input and outputs. Overfitting may lead to inadequate generalizations.

To mitigate this, the training set is supplemented with synthetic data specifically generated by GPT-4 to provide a challenge to the model by using sleep-related words and phrases in answers where context is key to understanding their meaning. This not only introduces diversity but also aids in the model's comprehension of complex relationships and contexts in sleep-related conversations. The BERT model is finely tuned to assess sleep quality on a nuanced one-five-star scale. The model takes a user's transcribed description of their sleep and outputs probabilities for all the five-star ratings. Originally trained on a broad range of general texts, including extensive internet resources, and then fine-tuned predicting the number of stars from online reviews, BERT's foundational training provides it with a deep understanding of language patterns and sentence structures (Muller, 2022). This is crucial for its initial capability in sentiment analysis.

The BERT model is fine-tuned with a specialized dataset that includes detailed user feedback specifically about sleep quality. This fine-tuning is pivotal in steering BERT's capabilities toward the unique requirements of sleep quality assessment. It enables the model to learn the specific language, idioms, and context related to sleep quality discussions, which are essential for accurate classification. By calibrating BERT with this sleep-specific dataset, the sleep analysis system significantly enhances the model's proficiency in assigning a precise one-to five-star rating to text inputs about sleep quality. This adaptation ensures that BERT not only grasps the general sentiment but also accurately interprets the subtle nuances that differentiate between the various levels of sleep quality.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations, or equivalents. It is intended that the following claims define the scope of the disclosure and that methods and structures within the scope of these claims and their equivalents be covered thereby.

What is claimed is:

1. A method, comprising:
(a) receiving a sleep dataset, at a patient interface, wherein the sleep dataset comprises at least: a plurality of user responses to a plurality of open-ended prompts corresponding to a sleep history of a user and at least one additional data modality, wherein the at least one additional data modality comprises data from a wearable sleep monitor;
(b) creating a user profile at a sleep analysis model based on the sleep dataset at a plurality of instances of time, the sleep analysis model comprising a symptom classifier;
(c) characterizing, at the symptom classifier, the plurality of user responses and the data from the wearable sleep monitor based at least in part on a set of sleep symptoms, to output a sleep symptom;
(d) generating, at the sleep analysis model, at least one insight based on a change in the user profile over the plurality of instances of time, wherein the at least one insight is indicative of the sleep symptom and is scored based on a metric of diagnostic value; and
(e) directing to a clinician interface a plurality of ranked action proposals based at least in part on the at least one insight and the metric of diagnostic value.

2. The method of claim 1, wherein the set of sleep symptoms, within the symptom classifier, is categorized as any one or any combination of insomnia, daytime sleepiness, daytime fatigue, abnormal breathing, snoring, waking often during sleep, movement abnormalities, restless leg syndrome, sleepwalking, sleep-eating, bedwetting, difficulty concentrating, nightmares, and needing stimulants to stay awake during daytime.

3. The method of claim 2, wherein a combination of symptoms of the set of sleep symptoms is indicative of a particular action.

4. The method of claim 1, further comprising: classifying the plurality of user responses based at least in part on a plurality of n-grams, wherein the plurality of n-grams is connected to an insight of the at least one insight.

5. The method of claim 4, further comprising: indicating at least one cause of a sleep disorder based at least in part on the plurality of n-grams.

6. The method of claim 1, wherein the plurality of open-ended prompts comprises human generated or AI-generated prompts in response to the plurality of user responses at the patient interface.

7. The method of claim 1, further comprising: annotating, at the patient interface, at least one of the plurality of ranked action proposals, wherein the patient interface is configured to generate at least one user-annotated ranked action proposal from a user annotation.

8. The method of claim 1, further comprising: annotating, at the clinician interface, at least one of the plurality of ranked action proposals, wherein the clinician interface is configured to generate at least one clinician-annotated ranked action proposal from a clinician annotation.

9. The method of claim 1, further comprising: communicating, at the patient interface, at least one of the plurality of ranked action proposals to the user, a clinician, or any combination thereof.

10. The method of claim 1, wherein the at least one additional data modality further comprises one or more of: CPAP machine data, spirometer data, non-sleep-medical data, or sleep ranking data.

11. The method of claim 1, wherein the wearable sleep monitor comprises a wearable device.

12. The method of claim 1, wherein the data from the wearable sleep monitor comprises one or more of pulse rate, oxygen saturation, or derived sleep stage.

13. The method of claim 1, wherein (d) comprises a proposal for a telehealth interaction.

14. The method of claim 1, further comprising: at (d) receiving from a clinician a text-based summary of a clinician analysis.

15. The method of claim 1, wherein the at least one insight comprises a pattern, trend, or anomaly in a user's sleep.

16. The method of claim 15, further comprising: thresholding the at least one insight based at least in part on a metric of diagnostic value.

17. The method of claim 1, further comprising: updating the user profile each time a new data point is added to the sleep dataset.

18. The method of claim 1, wherein, at (c), the symptom classifier comprises a language processing model, wherein the language processing model categorizes the plurality of user responses at least in part on the set of sleep symptoms.

19. The method of claim 1, wherein, at (c), the symptom classifier comprises a sleep-rating classifier, wherein the sleep-rating classifier ranks the plurality of user responses on an ordinal scale.

20. A system for sleep analysis, the system comprising:
a patient interface, the patient interface configured to receive a sleep dataset, wherein the sleep dataset comprises at least: a plurality of user responses to a plurality of open-ended prompts corresponding to a sleep history of a user and at least one additional data modality, wherein the at least one additional data modality comprises data from a wearable sleep monitor;
a sleep analysis system, the sleep analysis system configured to create a user profile based on the sleep dataset at a first instance of time, wherein the sleep analysis system is configured to generate at least one insight based on a change in the user profile from the first instance of time to a second instance of time, and wherein the sleep analysis system comprises:
a symptom classifier, wherein the symptom classifier is configured to categorize the plurality of user responses and the data from the wearable sleep monitor; and
a clinician interface, the clinician interface configured to provide a plurality of ranked action proposals based at least in part on the at least one insight.
